(19) United States
(12) Patent Application Publication
(10) Pub. No.: US 2024/0425452 A1
(43) Pub. Date: Dec. 26, 2024

(54) ISOLATED TRANS ISOMER OF 3-(2-BROMO-3,4-DIHYDROXY-PHENYL)-N-(3,4,5-TRIHYDROXY-BENZYL)-THIOACRYLAMIDE

(71) Applicant: TYRNOVO LTD., Rehovot (IL)

(72) Inventor: Hadas REUVENI, Har Adar (IL)

(73) Assignee: TYRNOVO LTD., Rehovot (IL)

(21) Appl. No.: 18/706,782

(22) PCT Filed: Oct. 3, 2022
(86) PCT No.: PCT/IL2022/051052

§ 371 Date: May 2, 2024

Related U.S. Application Data
(60) Provisional application No. 63/275,488, filed on Nov. 4, 2021.

(51) Int. Cl. C07C 327/44; A61K 9/08; A61K 31/165; A61K 45/06; A61K 47/40; A61P 35/00

(52) U.S. Cl. CPC C07C 327/44; A61K 9/08; A61K 31/165; A61K 45/06; A61K 47/40; A61P 35/00

(57) ABSTRACT

The present invention discloses an isolated substantially pure trans isomer of 3-(2-bromo-3,4-dihydroxy-phenyl)-N-(3,4,5-trihydroxy-benzyl)-thioacrylamide, methods for its preparation, pharmaceutical compositions comprising same, and use thereof in treating cancer.

### FIELD OF THE INVENTION

The present invention relates to an isolated trans isomer of 3-(2-bromo-3,4-dihydroxy-phenyl)-N-(3,4,5-trihydroxy-benzyl)-thioacrylamide and use thereof in treating cancer.

### BACKGROUND OF THE INVENTION

Cancer is a globally common disease, with a wide range of target sites in the tissues. It is characterized by uncontrolled proliferation of cells as a result of various factors, from contaminants to genetic predispositions. Current cancer therapy includes surgical intervention to remove solid tumors and systemic cancer therapy using chemotherapeutic agents, hormones, and immunotherapeutic agents. Although there is a variety of existing drugs effective in treating different types of cancer, in many instances the cancer becomes resistant to drugs. Accordingly, new therapeutic strategies are desired.

WO 2008/068751 discloses compounds having increased inhibitory properties of insulin-like growth factor 1 receptor (IGF1R), platelet derived growth factor receptor (PDGFR), epidermal growth factor receptor (EGFR), and IGF1R-related insulin receptor (IR) activation and signaling.

WO 2009/147682 discloses compounds acting as protein kinase (PK) and receptor kinase (RK) signaling modulators. Further disclosed in WO 2009/147682 are methods of preparation of the such compounds, pharmaceutical compositions including such compounds, and methods of using these compounds and compositions, especially as chemotherapeutic agents for preventions and treatments of PK and RK related disorders such as metabolic, inflammatory, fibrotic, and cell proliferative disorders, in particular cancer.

WO 2012/117396 describes combinations of the compounds of WO 2008/068751 or WO 2009/147682 with anti-cancer agents for the treatment of cancer.

WO 2016/125169 describes combinations of the compounds of WO 2008/068751 or WO 2009/147682 with (i) an Epidermal Growth Factor Receptor inhibitor (EGFR inhibitor) and EGFR antibody; (ii) an inhibitor of mammalian target of rapamycin (mTOR); (iii) a mitogen-activated protein kinase (MEK) inhibitor; (iv) a mutated B-Raf inhibitor; (v) an immunotherapy agent; and (vi) a chemotherapeutic agent, for the treatment of cancer.

WO 2019/097503 relates to the treatment of cancer using combination therapy comprising a dual modulator of Insulin Receptor Substrate (IRS) and signal transducer and activator of transcription 3 (Stat3), in combination with an antibody against programmed cell death 1 (PD-1) protein, an anti-programmed cell death protein 1 ligand (PD-L1) antibody, or a combination thereof. The combination can be used to re-sensitize a tumor that may develop or has developed resistance to the anti-PD-1 and/or anti-PD-L1 antibody, by enhancing response of the tumor to the anti-PD-1 and/or anti-PD-L1 antibody, converting non-responding tumors to responders and/or blocking tumor progression.

3-(2-bromo-3,4-dihydroxy-phenyl)-N-(3,4,5-trihydroxy-benzyl)-thioacrylamide, disclosed for the first time in WO 2009/147682 (compound 5), is a dual modulator of Insulin Receptor Substrate (IRS) and a signal transducer and activator of transcription 3 (Stat3). It includes a double bond conjugated to a thioamide bridging between two catechol rings. WO 2009/147682 teaches that this compound as well as other compounds disclosed therein can be in any structural and geometrical isomer, including inter alia, cis and trans isomers.

There is yet an unmet need for compounds with improved IRS and Stat3 inhibitory properties useful in the treatment of cancer.

### SUMMARY OF THE INVENTION

The present invention provides a substantially pure isolated trans isomer of 3-(2-bromo-3,4-dihydroxy-phenyl)-N-(3,4,5-trihydroxy-benzyl)-thioacrylamide, pharmaceutical compositions comprising same, and use thereof in the treatment of cancer.

It is now disclosed for the first time that a substantially pure trans isomer of 3-(2-bromo-3,4-dihydroxy-phenyl)-N-(3,4,5-trihydroxy-benzyl)-thioacrylamide provides improved anti-proliferative activity as compared to a cis and trans mixture of 3-(2-bromo-3,4-dihydroxy-phenyl)-N-(3,4,5-trihydroxy-benzyl)-thioacrylamide. It is further disclosed herein for the first time that a substantially pure trans isomer of 3-(2-bromo-3,4-dihydroxy-phenyl)-N-(3,4,5-trihydroxy-benzyl)-thioacrylamide exerts superior octanol:water partition coefficient as well as thermal behavior as compared to the cis isomer, which properties are indicative of its improved bioavailability. Contrary to conventional cis-trans stereoisomers which contain double bonds that do not rotate, it was unexpectedly found that rotation of the double bond in 3-(2-bromo-3,4-dihydroxy-phenyl)-N-(3,4,5-trihydroxy-benzyl)-thioacrylamide can occur freely upon exposure to light thereby affording interconversion between the trans and cis isomers in solution. It was unexpectedly shown that the anti-proliferative activity of the compound was reduced with increasing contents of the cis isomer due to light induced isomerization. It was further unexpectedly shown that the trans isomer was effective against cancer cells while the trans-cis mixture was less effective in the same concentrations. Accordingly, a substantially pure trans isomer is contemplated to have improved bioavailability and increased potency in the treatment of cancer as compared to the cis isomer or a trans-cis mixture containing more than 20% of the cis isomer.

According to a first aspect, the present invention provides a substantially pure trans isomer of 3-(2-bromo-3,4-dihydroxy-phenyl)-N-(3,4,5-trihydroxy-benzyl)-thioacrylamide having the following structural formula:

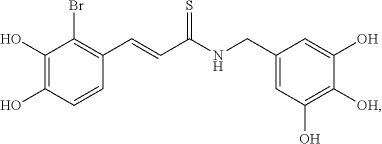 [SMILES: Oc1cc(CNC(=S)/C=C/c2ccc(O)c(O)c2Br)cc(O)c1O]

or a pharmaceutically acceptable salt thereof.

As used herein and unless otherwise indicated, the term “substantially pure” refers to the compound 3-(2-bromo-3,4-dihydroxy-phenyl)-N-(3,4,5-trihydroxy-benzyl)-thioacrylamide in a trans configuration which is substantially free of the cis isomer. For example, in some embodiments, a substantially pure trans isomer of the compound comprises greater than about 80% by weight of the trans isomer of the compound and less than about 20% by weight of the cis isomer of the compound. In other embodiments, a substantially pure trans isomer of the compound comprises greater than about 85% by weight of the trans isomer of the compound and less than about 15% by weight of the cis isomer of the compound. In yet other embodiments, a substantially pure trans isomer of the compound comprises greater than about 90% by weight of the trans isomer of the compound and less than about 10% by weight of the cis isomer of the compound. In currently preferred embodiments, a substantially pure trans isomer of the compound comprises greater than about 95% by weight of the trans isomer of the compound and less than about 5% by weight of the cis isomer of the compound. In additional currently preferred embodiments, a substantially pure trans isomer of the compound comprises greater than about 97% by weight of the trans isomer of the compound and less than about 3% by weight of the cis isomer of the compound.

According to some embodiments, the present invention provides a pharmaceutical composition comprising, as in active ingredient, a substantially pure trans isomer of 3-(2-bromo-3,4-dihydroxy-phenyl)-N-(3,4,5-trihydroxy-benzyl)-thioacrylamide or a pharmaceutically acceptable salt thereof, and a pharmaceutically acceptable carrier or excipient.

In certain embodiments, the pharmaceutical composition is in the form of a solution, suspension or emulsion. Each possibility represents a separate embodiment.

According to additional embodiments, the pharmaceutically acceptable carrier or excipient is hydroxypropyl-β-cyclodextrin (HPCD). According to further embodiments, the weight ratio between the substantially pure trans isomer of 3-(2-bromo-3,4-dihydroxy-phenyl)-N-(3,4,5-trihydroxy-benzyl)-thioacrylamide or a pharmaceutically acceptable salt thereof and the HPCD is from about 1:1 to about 1:12, including any ratio therebetween. According to particular embodiments, the weight ratio between the substantially pure trans isomer of 3-(2-bromo-3,4-dihydroxy-phenyl)-N-(3,4,5-trihydroxy-benzyl)-thioacrylamide or a pharmaceutically acceptable salt thereof and the HPCD is from about 1:4 to about 1:8, including any ratio therebetween.

According to some embodiments, the pharmaceutical composition is protected from visible light. In specific embodiments, the pharmaceutical composition is held in an apparatus which is impermeable to visible light. In further embodiments, the light impermeable apparatus has light transmission in the visible wavelength range of less than about 20%, preferably less than about 10%, and more preferably less than about 5%.

According to other embodiments, the pharmaceutical composition is stored in a container which is impermeable to visible light. In particular embodiments, the light impermeable container has light transmission in the visible wavelength range of less than about 20%, preferably less than about 10%, and more preferably less than about 5%.

According to further embodiments, the pharmaceutical composition is provided in a kit suitable for intravenous administration, wherein the kit is impermeable to visible light. In specific embodiments, the light impermeable kit has light transmission in the visible wavelength range of less than about 20%, preferably less than about 10%, and more preferably less than about 5%.

According to some embodiments, the pharmaceutical composition is protected from UV light. In specific embodiments, the pharmaceutical composition is held in an apparatus which is impermeable to UV light. In further embodiments, the light impermeable apparatus has light transmission in the UV wavelength range of less than about 20%, preferably less than about 10%, and more preferably less than about 5%.

According to other embodiments, the pharmaceutical composition is stored in a container which is impermeable to UV light. In particular embodiments, the light impermeable container has light transmission in the UV wavelength range of less than about 20%, preferably less than about 10%, and more preferably less than about 5%.

According to further embodiments, the pharmaceutical composition is provided in a kit suitable for intravenous administration wherein the kit is impermeable to UV light. In specific embodiments, the light impermeable kit has light transmission in the UV wavelength range of less than about 20%, preferably less than about 10%, and more preferably less than about 5%.

According to some embodiments, the pharmaceutical composition is useful in treating cancer. In certain embodiments, the present invention provides a method of treating cancer, the method comprising administering to a subject in need thereof the substantially pure trans isomer as disclosed herein or a pharmaceutical composition comprising same. In other embodiments, the present invention provides the use of a substantially pure trans isomer as disclosed herein for the preparation of a medicament for treating cancer. In further embodiments, the cancer is selected from the group consisting of head and neck cancer, sarcoma, multiple myeloma, ovarian cancer, breast cancer, kidney cancer, stomach cancer, hematopoietic cancers, lymphoma, leukemia, lung carcinoma, melanoma, glioblastoma, hepatocarcinoma, esophageal cancer, gastroesophageal junction cancer, prostate cancer, pancreatic cancer, and colon cancer. Each possibility represents a separate embodiment.

According to other embodiments, the substantially pure trans isomer of the present invention or a pharmaceutical composition comprising same is co-administered in combination with an anti-cancer agent. According to some embodiments, the anti-cancer agent comprises at least one of (i) a modulator of a protein kinase (PK) selected from an Epidermal Growth Factor Receptor inhibitor (EGFR inhibitor) and an EGFR antibody; (ii) an inhibitor of mammalian target of rapamycin (mTOR); (iii) a mitogen-activated protein kinase (MEK) inhibitor; (iv) a mutated B-Raf inhibitor; (v) a chemotherapeutic agent; and (vi) an immunotherapy agent comprising an antibody against programmed cell death 1 (PD-1) protein, programmed cell death protein 1 ligand (PD-L1), cytotoxic T-lymphocyte-associated protein 4 (CTLA4), or a combination thereof. Each possibility represents a separate embodiment. According to particular embodiments, co-administration is performed simultaneously or sequentially, in any order. Each possibility represents a separate embodiment.

According to additional embodiments, a method is provided for preventing the conversion of a substantially pure trans isomer of 3-(2-bromo-3,4-dihydroxy-phenyl)-N-(3,4,5-trihydroxy-benzyl)-thioacrylamide to the cis isomer, the method comprising maintaining the substantially pure trans isomer in a light-protected apparatus or container. In some embodiments, the light-protected apparatus or container is substantially impermeable to light in the UV-VIS wavelength range.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

The present invention will be more fully understood from the following figures and detailed description of the preferred embodiments thereof.

### BRIEF DESCRIPTION OF THE FIGURES

FIG. 1. Chemical structure with atom annotation of the trans isomer of 3-(2-bromo-3,4-dihydroxy-phenyl)-N-(3,4,5-trihydroxy-benzyl)-thioacrylamide.

FIG. 2. Chemical structure with atom annotation of the cis isomer of 3-(2-bromo-3,4-dihydroxy-phenyl)-N-(3,4,5-trihydroxy-benzyl)-thioacrylamide.

FIG. 3. ¹H NMR spectrum of a trans and cis mixture of 3-(2-bromo-3,4-dihydroxy-phenyl)-N-(3,4,5-trihydroxy-benzyl)-thioacrylamide after UV light exposure.

FIGS. 4A-4B. UV spectra (obtained from HPLC peaks) of the trans (4A) and the cis (4B) isomers of 3-(2-bromo-3,4-dihydroxy-phenyl)-N-(3,4,5-trihydroxy-benzyl)-thioacrylamide.

FIGS. 5A-5F. HPLC chromatograms of short-term stability tests of 3-(2-bromo-3,4-dihydroxy-phenyl)-N-(3,4,5-trihydroxy-benzyl)-thioacrylamide solutions: (5A) solution X1; (5B) solution X2; (5C) solution Y1; (5D) solution Y2; (5E) solution Z1; and (5F) solution Z2.

FIG. 6. Anti-proliferative activity in human melanoma A375 cells of solutions with different ratios of the cis:trans isomers of 3-(2-bromo-3,4-dihydroxy-phenyl)-N-(3,4,5-trihydroxy-benzyl)-thioacrylamide.

FIGS. 7A-7D. The partition coefficient between water and octanol to determine lipophilicity (Log P) of each isomer. HPLC chromatograms of: (7A) the cis isomer in the octanol phase; (7B) the cis isomer in the water phase; (7C) the trans isomer in the octanol phase; and (7D) the trans isomer in the water phase. Equal volumes (13 μL) of each phase were analyzed.

FIGS. 8A-8B. Stability of the cis and trans isomers in (8A) FASSGF; and (8B) FASSIF media.

FIGS. 9A-9C. Thermogravimetric analysis spectra of the (9A) trans isomer; and (9B) cis isomer of 3-(2-bromo-3,4-dihydroxy-phenyl)-N-(3,4,5-trihydroxy-benzyl)-thioacrylamide. (9C) An overlay of the spectra of the two isomers.

### DETAILED DESCRIPTION OF THE INVENTION

The present invention is directed to a substantially pure trans isomer of 3-(2-bromo-3,4-dihydroxy-phenyl)-N-(3,4,5-trihydroxy-benzyl)-thioacrylamide having the following structural formula:

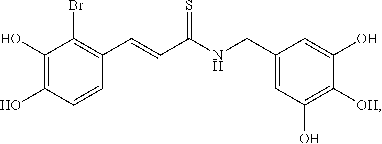 [SMILES: Oc1cc(CNC(=S)/C=C/c2ccc(O)c(O)c2Br)cc(O)c1O]

or a pharmaceutically acceptable salt thereof.

The present invention is further directed to a pharmaceutical composition comprising, as an active ingredient, a substantially pure trans isomer of 3-(2-bromo-3,4-dihydroxy-phenyl)-N-(3,4,5-trihydroxy-benzyl)-thioacrylamide or a pharmaceutically acceptable salt thereof, and a pharmaceutically acceptable carrier or excipient.

The present invention is further directed to methods of use of the substantially pure trans isomer of 3-(2-bromo-3,4-dihydroxy-phenyl)-N-(3,4,5-trihydroxy-benzyl)-thioacrylamide or a pharmaceutically acceptable salt thereof, or a pharmaceutical composition comprising same in treating cancer.

The present invention is based, in part, on the surprising discovery that contrary to conventional double bonds which do not allow free interconversion between cis and trans isomers, the double bond in 3-(2-bromo-3,4-dihydroxy-phenyl)-N-(3,4,5-trihydroxy-benzyl)-thioacrylamide can freely interconvert from the trans isomer to the cis isomer when exposed to UV-VIS light. While the substantially pure trans isomer showed high efficacy in inhibiting the proliferation of human melanoma A375 cells, a mixture of both isomers showed lower efficacy in inhibiting A375 cell proliferation. Thus, a substantially pure trans isomer of 3-(2-bromo-3,4-dihydroxy-phenyl)-N-(3,4,5-trihydroxy-benzyl)-thioacrylamide provides enhanced anti-proliferative efficacy as compared to a mixture containing both the trans and cis isomers. The substantially pure trans isomer was further characterized for its octanol:water partition coefficient, stability in biorelevant fluids, and thermal behavior. It is now disclosed for the first time that the substantially pure trans isomer is more stable in gastric and intestinal fluids and is indicated to exert improved bioavailability as compared to the cis isomer.

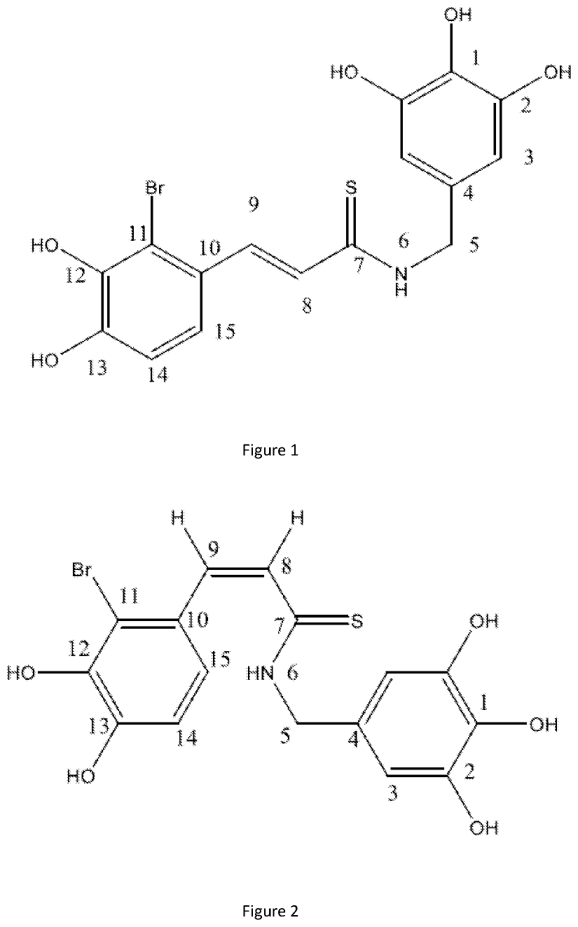

According to the principles of the present invention, the substantially pure trans isomer of 3-(2-bromo-3,4-dihydroxy-phenyl)-N-(3,4,5-trihydroxy-benzyl)-thioacrylamide comprises at least 80% by weight of the trans isomer as depicted in FIG. 1 and less than 20% by weight of the cis isomer as depicted in FIG. 2. Preferably, the substantially pure trans isomer of 3-(2-bromo-3,4-dihydroxy-phenyl)-N-(3,4,5-trihydroxy-benzyl)-thioacrylamide comprises at least 85% by weight of the trans isomer as depicted in FIG. 1 and less than 15% by weight of the cis isomer as depicted in FIG. 2. More preferably, the substantially pure trans isomer of 3-(2-bromo-3,4-dihydroxy-phenyl)-N-(3,4,5-trihydroxy-benzyl)-thioacrylamide comprises at least 90% by weight of the trans isomer as depicted in FIG. 1 and less than 10% by weight of the cis isomer as depicted in FIG. 2. Even more preferably, the substantially pure trans isomer of 3-(2-bromo-3,4-dihydroxy-phenyl)-N-(3,4,5-trihydroxy-benzyl)-thioacrylamide comprises at least 95% by weight of the trans isomer as depicted in FIG. 1 and less than 5% by weight of the cis isomer as depicted in FIG. 2. Most preferably, the substantially pure trans isomer of 3-(2-bromo-3,4-dihydroxy-phenyl)-N-(3,4,5-trihydroxy-benzyl)-thioacrylamide comprises at least 97% by weight of the trans isomer as depicted in FIG. 1 and less than 3% by weight of the cis isomer as depicted in FIG. 2.

The synthesis of a substantially pure trans isomer of 3-(2-bromo-3,4-dihydroxy-phenyl)-N-(3,4,5-trihydroxy-benzyl)-thioacrylamide can be performed as is known in the art. Briefly, the synthesis involves the following steps: First, 2-bromo-3,4-dimethoxybenzaldehyde and malonic acid are reacted in Knoevenagel condensation to give 2-bromo-3,4-dimethoxycinnamic acid. Second, 2-bromo-3,4-dimethoxycinnamic acid is then transformed to an acyl chloride derivative which is further reacted with 3,4,5-trimethoxybenzylamine to give 3-(2-bromo-3,4-dimethoxy-benzyl)-N-(3,4,5-dimethoxy-benzyl)-acrylamide. Third, Lawesson's reagent is then used as a thiation agent to give 3-(2-bromo-3,4-dimethoxy-benzyl)-N-(3,4,5-dimethoxy-benzyl)-thio-acrylamide from the previous compound. Finally, boron tribromide is used to cleavage the methoxy protecting groups to give the final product.

According to some aspects and embodiments, the substantially pure trans isomer of 3-(2-bromo-3,4-dihydroxy-phenyl)-N-(3,4,5-trihydroxy-benzyl)-thioacrylamide has physical properties such that it remains stable in a gastric environment for at least 2 hours. In other aspects and embodiments, the substantially pure trans isomer of 3-(2-bromo-3,4-dihydroxy-phenyl)-N-(3,4,5-trihydroxy-benzyl)-thioacrylamide has physical properties such that it remains stable in a gastric environment for at least 3 hours. In yet other aspects and embodiments, the substantially pure trans isomer of 3-(2-bromo-3,4-dihydroxy-phenyl)-N-(3,4,5-trihydroxy-benzyl)-thioacrylamide has physical properties such that it remains stable in a gastric environment for at least 4 hours. In further aspects and embodiments, the substantially pure trans isomer of 3-(2-bromo-3,4-dihydroxy-phenyl)-N-(3,4,5-trihydroxy-benzyl)-thioacrylamide has physical properties such that it remains stable in an intestinal environment for at least 2 hours. In other aspects and embodiments, the substantially pure trans isomer of 3-(2-bromo-3,4-dihydroxy-phenyl)-N-(3,4,5-trihydroxy-benzyl)-thioacrylamide has physical properties such that it remains stable in an intestinal environment for at least 3 hours. In yet other aspects and embodiments, the substantially pure trans isomer of 3-(2-bromo-3,4-dihydroxy-phenyl)-N-(3,4,5-trihydroxy-benzyl)-thioacrylamide has physical properties such that it remains stable in an intestinal environment for at least 4 hours. As used herein, the term “stable” refers to a decrease in concentration of less than about 10% of the initial concentration.

The substantially pure trans isomer of the invention may be present as a pharmaceutically acceptable salt thereof. The term “salt” encompasses both basic and acid addition salts including, but not limited to, carboxylate salts or salts with amine nitrogens, and include salts formed with the organic and inorganic anions and cations discussed below. Furthermore, the term includes salts that are formed by standard acid-base reactions with basic groups (such as amino groups) and organic or inorganic acids. Such acids include hydrochloric, hydrofluoric, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicylic, methanesulfonic, benzenesulfonic, sorbic, picric, benzoic, cinnamic, and like acids. Each possibility represents a separate embodiment of the invention.

The term “organic or inorganic cation” refers to counter-ions for the anion of a salt. The counter-ions include, but are not limited to, alkali and alkaline earth metals (such as lithium, sodium, potassium, barium, aluminum and calcium); ammonium and mono-, di- and tri-alkyl amines such as trimethylamine, cyclohexylamine; and the organic cations, such as dibenzylammonium, benzylammonium, 2-hydroxyethylammonium, bis(2-hydroxyethyl)ammonium, phenylethylbenzylammonium, dibenzylethylenediammonium, and the like. See, for example, Berge et al., J. Pharm. Sci. (1977), 66:1-19, which is incorporated herein by reference.

Within the scope of the present invention are pharmaceutical compositions comprising the substantially pure trans isomer of the invention or a pharmaceutically acceptable salt thereof, and a pharmaceutically acceptable carrier or excipient. The pharmaceutical compositions may be in the form of liquid preparations such as, but not limited to, liquid solutions, suspensions, or emulations or in the form of solid preparations such as, but not limited to, tablets, powders, capsules, or pellets. Each possibility represents a separate embodiment.

In order to avoid the interconversion of the substantially pure trans isomer to the cis isomer, the pharmaceutical composition is preferably shielded from visible and/or UV light e.g. using a light resistant apparatus and/or container. Suitable light-shielding is performed with any of the followings or combinations thereof: use of amber containers (e.g. amber glass crimp top vials), aluminum foil coverage, black plastic coverage, dark room, double sleeve jacket, covered infusion kit, etc. Each possibility represents a separate embodiment. Specifically, the light resistant apparatus, container or coverage has light transmission of less than about 20%, preferably less than about 10%, and more preferably less than about 5% in the UV-VIS wavelength range.

Thus, the present invention further comprises a method of preventing the conversion of a substantially pure trans isomer of 3-(2-bromo-3,4-dihydroxy-phenyl)-N-(3,4,5-trihydroxy-benzyl)-thioacrylamide to the cis isomer of 3-(2-bromo-3,4-dihydroxy-phenyl)-N-(3,4,5-trihydroxy-benzyl)-thioacrylamide, the method comprising maintaining the substantially pure trans isomer or a composition comprising same in a light resistant apparatus, container or coverage as detailed above. According to some embodiments, the light-resistant apparatus, container or coverage are substantially impermeable to light in the UV-VIS wavelength range. The term “light impermeable”, as used herein refers to apparatus, container or coverage that at least partially or preferably completely prevent light transmission in a wavelength range of about 100 to about 800 nanometers (nm), including each value within the specified range. In some embodiments, the light impermeable apparatus, container or coverage at least partially or preferably completely prevent light transmission in the UV wavelength range of about 100 to about 400 nanometers (nm), including each value within the specified range. In other embodiments, the light impermeable apparatus, container or coverage at least partially or preferably completely prevent light transmission in the visible wavelength range of about 400 to about 800 nanometers (nm), including each value within the specified range. Typically, the light transmission in the UV and/or visible range is less than about 20%, 15%, 10%, 5%, or 1% with each possibility representing a separate embodiment.

Within the scope of the present invention is the optional inclusion of at least one pharmaceutically acceptable carrier or excipient in the pharmaceutical compositions disclosed herein. Suitable pharmaceutically acceptable carriers or excipients include, but are not limited to, diluents, preservatives, solubilizers, emulsifiers, adjuvants and the like. Each possibility represents a separate embodiment. Such compositions are liquids or lyophilized or otherwise dried formulations and include diluents of various buffer content (e.g., Tris-HCl, acetate, phosphate), pH and ionic modifiers, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), and bulking substances or tonicity modifiers (e.g., lactose, mannitol). Each possibility represents a separate embodiment.

Moreover, as used herein “pharmaceutically acceptable carriers” are well known to those skilled in the art and include, but are not limited to, 0.01-0.1M and preferably 0.05M phosphate buffer or 0.8% saline. Additionally, such pharmaceutically acceptable carriers may be aqueous or non-aqueous solutions, suspensions, and emulsions. Each possibility represents a separate embodiment. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Each possibility represents a separate embodiment.

Parenteral vehicles include sodium chloride solution, Ringer's dextrose, dextrose and sodium chloride, lactated Ringer's or fixed oils. Each possibility represents a separate embodiment. Intravenous vehicles include fluid and nutrient replenishers, electrolyte replenishers such as those based on Ringer's dextrose, and the like. Preservatives and other additives may also be present, such as, for example, antimicrobials, antioxidants, and the like. Each possibility represents a separate embodiment.

Controlled or sustained release compositions include formulation in lipophilic depots (e.g. fatty acids, waxes, oils). Also comprehended by the invention are particulate compositions coated with polymers (e.g. poloxamers or poloxamines) and protective coatings.

Pharmaceutically acceptable carriers may further include gums, starches, sugars, cellulosic materials, lactose, acacia, gelatin, alginic acid, stearic acid or magnesium stearate. Each possibility represents a separate embodiment.

Examples of suitable oily vehicles or solvents are vegetable or animal oils such as sunflower oil or fish-liver oil, petroleum, or oil derived from animal, vegetable, or synthetic origin, for example, peanut oil, soybean oil, or mineral oil. Each possibility represents a separate embodiment. Examples of suitable water-based vehicles are water, saline, aqueous dextrose and related sugar solutions, and glycols such as propylene glycols or polyethylene glycols, glycerol or ethanol. Each possibility represents a separate embodiment. In addition, if desired, the composition can contain minor amounts of auxiliary substances such as wetting or emulsifying agents and/or pH buffering agents.

In currently preferred embodiments, the pharmaceutical compositions disclosed herein contain a chelating agent. Suitable chelating agents within the scope of the present invention include, but are not limited to, cyclodextrin (modified or unmodified) such as, but not limited to, α-cyclodextrin, β-cyclodextrin, γ-cyclodextrin, 2-hydroxypropyl-β-cyclodextrin, methyl-β-cyclodextrin, sulfobutylether β-cyclodextrin, and a mixture or combination thereof. Each possibility represents a separate embodiment. In currently preferred embodiments, the chelating agent is hydroxypropyl-β-cyclodextrin (HPCD). Typically, a chelating agent such as HPCD is present in the composition in an amount resulting in an about 1:1 to about 1:12 weight ratio between the substantially pure trans isomer and the HPCD. Suitable weight ratios between the substantially pure trans isomer and the HPCD within the scope of the present invention include, but are not limited to, about 1:1, about 1:2, about 1:3, about 1:4, about 1:5, about 1:6, about 1:7, about 1:8, about 1:9, about 1:10, about 1:11, or about 1:12, with each possibility representing a separate embodiment. According to particular embodiments, the weight ratio between the substantially pure trans isomer and the HPCD is from about 1:4 to about 1:8, e.g. about 1:6.

According to the principles of the present invention, the substantially pure trans isomer or a composition comprising same is useful for treating cancer.

Thus, the present invention provides a method of treating cancer comprising administering to a subject in need thereof a therapeutically effective amount of a substantially pure trans isomer of 3-(2-bromo-3,4-dihydroxy-phenyl)-N-(3,4,5-trihydroxy-benzyl)-thioacrylamide or a pharmaceutical composition comprising same.

“A therapeutically effective amount” as used herein refers to an amount of an agent which is effective, upon single or multiple dose administrations to the subject in providing a therapeutic benefit to the subject. In one embodiment, the therapeutic benefit is the treatment of cancer. The amount that will be effective in treatment will depend on the nature of the disorder or condition, and can be determined by standard clinical techniques. In addition, in vitro assays may optionally be employed to help identify optimal dose ranges. The precise dose to be employed will also depend on the route of administration, and the progression of the disease or disorder, and should be decided according to the judgment of the practitioner and each patient's circumstances. A preferred dosage will be within the range of 0.01 mg/kg to 1000 mg/kg of body weight, 0.1 mg/kg to 100 mg/kg, 1 mg/kg to 100 mg/kg, 10 mg/kg to 75 mg/kg, 0.1 mg/kg to 1 mg/kg, etc., including each value within the specified ranges. Exemplary, non-limiting amounts include 0.1 mg/kg, 0.2 mg/kg, 0.5 mg/kg, 1 mg/kg, 5 mg/kg, 10 mg/kg, 20 mg/kg, 50 mg/kg, 60 mg/kg, 75 mg/kg, and 100 mg/kg. Each possibility represents a separate embodiment.

Alternatively, the amount administered can be measured and expressed as molarity of the administered compound. By way of illustration and not limitation, the substantially pure trans isomer of 3-(2-bromo-3,4-dihydroxy-phenyl)-N-(3,4,5-trihydroxy-benzyl)-thioacrylamide can be administered in a range of 0.1-10 mM, e.g., 0.1, 0.25, 0.5, 1, and 2 mM. Each possibility represents a separate embodiment. Alternatively, the amount administered can be measured and expressed as mg/ml, μg/ml, or ng/ml. By way of illustration and not limitation, typical amounts include 1 ng/ml to 1,000 mg/ml, for example 1-1,000 ng/ml, 1-100 ng/ml, 1-1,000 μg/ml, 1-100 μg/ml, 1-1,000 mg/ml, 1-100 mg/ml, etc., including each value within the specified ranges. Effective doses may be extrapolated from dose-response curves derived from in vitro or animal model test bioassays or systems.

The term “treatment of cancer” in the context of the present invention includes at least one of the following: a decrease in the rate of growth of the cancer (i.e., the cancer still grows but at a slower rate); cessation of growth of the cancer, i.e., stasis of the tumor growth, and, in preferred cases, the tumor diminishes or is reduced in size. The term also includes reduction in the number of metastases, reduction in the number of new metastases formed, slowing of the progression of cancer from one stage to the other and a decrease in the angiogenesis induced by the cancer. In most preferred cases, the tumor is totally eliminated. Additionally included in this term is lengthening of the survival period of the subject undergoing treatment, lengthening the time of disease progression, tumor regression, and the like. It is to be understood that the term “treating cancer” also refers to the inhibition of a malignant (cancer) cell proliferation including tumor formation, primary tumors, tumor progression or tumor metastasis. The term “inhibition of proliferation” in relation to cancer cells, may further refer to a decrease in at least one of the following: number of cells (due to cell death which may be necrotic, apoptotic or any other type of cell death or combinations thereof) as compared to control; decrease in growth rates of cells, i.e. the total number of cells may increase but at a lower level or at a lower rate than the increase in control; decrease in the invasiveness of cells (as determined for example by soft agar assay) as compared to control even if their total number has not changed; progression from a less differentiated cell type to a more differentiated cell type; a deceleration in the neoplastic transformation; or alternatively the slowing of the progression of the cancer cells from one stage to the next.

The term “cancer” as used herein refers to a disorder in which a population of cells has become, in varying degrees, unresponsive to the control mechanisms that normally govern proliferation and differentiation. Cancer refers to various types of malignant neoplasms and tumors, including primary tumors, and tumor metastasis. Non-limiting examples of cancers which can be treated by the substantially pure trans isomer or pharmaceutical compositions comprising same are brain, ovarian, colon, prostate, kidney, bladder, breast, lung, oral, and skin cancers. Each possibility represents a separate embodiment. Specific examples of cancers are: carcinomas, sarcomas, myelomas, leukemias, lymphomas and mixed type tumors. Each possibility represents a separate embodiment. Particular categories of tumors include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Each possibility represents a separate embodiment. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, squamous cell carcinoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma, and hepatocarcinoma. Each possibility represents a separate embodiment.

In some representative embodiments, the cancer is selected from the group consisting of head and neck (H&N) cancer, sarcoma, multiple myeloma, ovarian cancer, breast cancer, kidney cancer, stomach cancer, hematopoietic cancers, lymphoma, leukemia (including lymphoblastic leukemia), lung carcinoma, melanoma, glioblastoma, hepatocarcinoma, esophageal cancer, gastroesophageal junction cancer, prostate cancer, and colon cancer. Each possibility represents a separate embodiment.

Routes of administration include, but are not limited to, oral, topical, transdermal, intra-arterial, intranasal, intraperitoneal, intramuscular, subcutaneous, intravenous, intratracheal, intrabronchial, intra-alveolar, transmucosal, intraventricular, intracranial and intratumoral. Each possibility represents a separate embodiment. The present invention further provides the administration of the substantially pure trans isomer of 3-(2-bromo-3,4-dihydroxy-phenyl)-N-(3,4,5-trihydroxy-benzyl)-thioacrylamide in combination therapy with at least one additional anti-cancer agent. Such additional anti-cancer agents according to the principles of the present invention include, but are not limited to, Epidermal Growth Factor Receptor inhibitors (EGFR inhibitors) including erlotinib, gefitinib, lapatinib, vandetanib, neratinib, icotinib, afatinib, dacomitinib, poziotinib, AZD9291, CO-1686, HM61713 and AP26113; EGFR antibodies including trastuzumab, cetuximab, necitumumab and panitumumab; Mammalian Target of Rapamycin inhibitors (mTOR inhibitors) including rapamycin (Sirolimus), Ridaforolimus (AP23573), NVP-BEZ235, Everolimus (Afinitor, RAD-001), Temsirolimus (CCI-779), OSI-027, XL765, INK128, MLN0128, AZD2014, DS-3078a and Palomid529; Mitogen-activated protein kinase inhibitors (MEK inhibitors) including Trametinib (GSK1120212), Selumetinib, Binimetinib (MEK162), PD-325901, Cobimetinib, CI-1040 and PD035901; Mutated B-Raf inhibitors including Vemurafenib (PLX-4032), PLX4720, Sorafenib (BAY43-9006), and Dabrafenib; chemotherapeutic agents including topoisomerase inhibitors, spindle poison vincas: vinblastine, vincristine, vinorelbine (taxol), paclitaxel, docetaxel; and alkylating agents: mechlorethamine, chlorambucil, cyclophosphamide, melphalan, ifosfamide; methotrexate; 6-mercaptopurine; 5-fluorouracil, cytarabine, gemcitabine; podophyllotoxins: etoposide, irinotecan, topotecan; anticancer chemicals containing a quinone group: carbazilquinone; antibiotics: doxorubicin (adriamycin), daunorubicin, idarubicin, epirubicin, bleomycin, mitomycin; nitrosoureas: carmustine (BCNU), lomustine; inorganic ions: cisplatin, carboplatin, and oxaliplatin; interferon, asparaginase; hormones: tamoxifen, leuprolide, flutamide, and megestrol acetate, and dacarbazine; and an immunotherapy agent comprising an antibody against a target selected from the group consisting of programmed cell death protein 1 (PD-1), programmed cell death protein 1 ligand (PD-L1), and cytotoxic T-lymphocyte-associated protein 4 (CTLA4) including Pembrolizumab (Keytruda), Nivolumab (Opdivo), AGEN-2034, AMP-224, BCD-100, BGBA-317, BI-754091, CBT-501, CC-90006, Cemiplimab, GLS-010, IBI-308, JNJ-3283, JS-001, MEDI-0680, MGA-012, MGD-013, PDR-001, PF-06801591, REGN-2810, SHR-1210, TSR-042, LZM-009, ABBV-181, Pidilizumab, Avelumab (Bavencio), Durvalumab (Imfinzi), Atezolizumab (Tecentriq), BMS-936559, CX-072, SHR-1316, M-7824, LY-3300054, FAZ-053, KN-035, CA-170, CK-301, CS-1001, HLX-10, MCLA-145, MSB-2311, and MEDI-4736. Each possibility represents a separate embodiment.

Within the scope of the combination therapy of the present invention are combinations with immunotherapy agents which are antibodies against a target comprising any of CD20, CD30, CD33, CD52, VEGF, and ErbB2. Each possibility represents a separate embodiment.

It is further contemplated that the combination therapy will include the two or more active ingredients within a single pharmaceutical composition as well as in two separate pharmaceutical compositions administered to the same subject simultaneously or at a time interval determined by a skilled artisan. For example, administration of a pharmaceutical composition disclosed herein can take place prior to, after or at the same time as the administration of the other anti-cancer agent. The other anti-cancer agent(s) can be administered prior to onset of treatment with the pharmaceutical composition disclosed herein or following treatment with the pharmaceutical composition disclosed herein. In addition, the other anti-cancer agent(s) can be administered during the period of administration of the pharmaceutical composition disclosed herein but does not need to occur over the entire treatment period. In another embodiment, the treatment regimen includes pre-treatment with one agent, either the pharmaceutical composition disclosed herein or the other anti-cancer agent, followed by the addition of the other agent or agents. Alternating sequences of administration are also contemplated as are known in the art.

As used herein and in the appended claims, the term “about” refers to ±10%.

As used herein and in the appended claims, the singular forms “a”, “an”, and “the” include plural references unless the context clearly dictates otherwise. Thus, for example, reference to “an anti-cancer agent” includes a plurality of such agents. It should be noted that the term “and” or the term “or” are generally employed in their sense including “and/or” unless the context clearly dictates otherwise.

The principles of the present invention are demonstrated by means of the following non-limiting examples.

### EXAMPLES

### Example 1: Synthesis of the Trans Isomer of 3-(2-bromo-3,4-dihydroxy-phenyl)-N-(3,4,5-trihydroxy-benzyl)-thioacrylamide

All chemicals used for chemical synthesis were purchased from Sigma.

### Synthesis of 2-bromo-3,4-dimethoxycinnamic acid

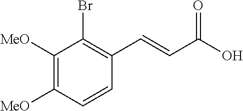 [SMILES: COc1ccc(/C=C/C(=O)O)c(Br)c1OC]

A catalytic amount of piperidine (0.2 equiv.) was added to a solution of 2-bromo-3,4-dimethoxybenzaldehyde (1 equiv.) and malonic acid (1.5 equiv.) in pyridine (4 ml/mmol aldehyde). The reaction mixture was heated to 120° C. for 6 hours. The solution was cooled to 0° C. and concentrated HCl was added drop-wise to a pH<3. The precipitate was collected by filtration, washed with water and dried under reduced pressure to give 2-bromo-3,4-dimethoxycinnamic acid in 62% yield as a white solid. ¹H NMR (400 MHz, CDCl₃+ Acetone-d₆): δ 8.07 (d, J=15.6 Hz), 7.45 (d, J=8.8 Hz, 1H), 6.95 (d, J=8.8 Hz, 1H), 6.31 (d, J=15.6 Hz, 1H), 3.93 (s, 3H), 3.85 (s, 3H).

### Synthesis of the Trans Isomer of 3-(2-bromo-3,4-dimethoxyphenyl)-N-(3,4,5-trimethoxy-benzyl)-acrylamide

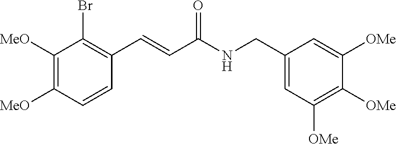 [SMILES: COc1cc(CNC(=O)/C=C/c2ccc(OC)c(OC)c2Br)cc(OC)c1OC]

Oxalyl chloride (4 equiv.) was added to a cooled solution of 2-bromo-3,4-dimethoxycinnamic acid (1 equiv.) in CH₂Cl₂, and the solution was stirred for 1-2 hours at room temperature. The excess of oxalyl chloride was distilled off and the mixture was evaporated to dryness. The residue was dissolved in CH₂Cl₂ and added drop-wise to a cooled solution of 3,4,5-trimethoxybenzylamine (0.9 equiv.) and Et₃N (4 equiv.) in CH₂Cl₂. The reaction mixture was stirred at room temperature overnight (until TLC indicated the disappearance of the amine) and then treated with water. The CH₂Cl₂ was evaporated under reduced pressure and the residue was filtered and washed with ethyl acetate. The filtrate was extracted twice with ethyl acetate and the combined organic phases were dried over Na₂SO₄, filtered and the solvent was evaporated to give brown solid. The crude solid was purified by flash chromatography (ethyl acetate/hexane) to give 3-(2-bromo-3,4-dimethoxy-benzyl)-N-(3,4,5-trimethoxyphenyl-benzyl)-acrylamide in 55% yield as a white solid. ¹H NMR (400 MHz, CDCl₃): δ 7.96 (d, J=15.6 Hz, 1H), 7.31 (d, J=8.8 Hz, 1H), 6.86 (d, J=8.8 Hz, 1H), 6.55 (s, 2H), 6.28 (d, J=15.6 Hz, 1H), 5.91 (bt, 1H), 4.50 (d, J=5.6 Hz, 2H), 3.90 (s, 3H), 3.85 (s, 9H), 3.84 (s, 3H).

### Synthesis of the Trans Isomer of 3-(2-bromo-3,4-dimethoxyphenyl)-N-(3,4,5-trimethoxy-benzyl)-thioacrylamide

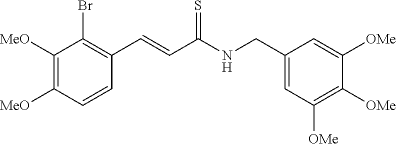 [SMILES: COc1cc(CNC(=S)/C=C/c2ccc(OC)c(OC)c2Br)cc(OC)c1OC]

3-(2-bromo-3,4-dimethoxy-benzyl)-N-(3,4,5-trimethoxyphenyl)-acrylamide (1 equiv.) and Lawesson's reagent (0.55 equiv.) were refluxed in toluene for 3 hours (until TLC indicated the disappearance of the amide). The reaction mixture was cooled to room temperature. The crude mixture was adsorbed onto silica gel and purified by column chromatography (ethyl acetate/hexane) to yield 3-(2-bromo-3,4-dimethoxy-benzyl)-N-(3,4,5-trimethoxyphenyl)-thioacrylamide in 50% yield as a pale yellow solid. ¹H NMR (400 MHz, CDCl₃): δ 8.05 (d, J=15.6 Hz, 1H), 7.45 (bt, 1H), 7.33 (d, J=8.8 Hz, 1H), 6.86 (d, J=8.8 Hz, 1H), 6.73 (d, J=15.6 Hz, 1H), 6.60 (s, 2H), 4.89 (d, J=5.2 Hz, 2H), 3.91 (s, 3H), 3.86 (s, 6H), 3.85 (s, 3H), 3.83 (s, 3H).

### Synthesis of the Trans Isomer of 3-(2-bromo-3,4-dihydroxyphenyl)-N-(3,4,5-trihydroxy-benzyl)-thioacrylamide

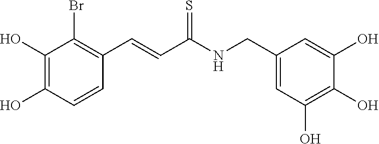 [SMILES: Oc1cc(CNC(=S)/C=C/c2ccc(O)c(O)c2Br)cc(O)c1O]

Boron tribromide (2.5 equiv. excess for each methoxy group) was added to an ice-cold solution of 3-(2-bromo-3,4-dimethoxyphenyl)-N-(3,4,5-trimethoxy-benzyl)-thioacrylamide in CH₂Cl₂ (ca. 20 ml/mmol). The reaction mixture was allowed to warm to room temperature and stirred for 5 hours. The solution was cooled to 0° C. and then treated with cooled water. The DCM was evaporated and the solution was extracted three times with ethyl acetate. The combined organic layers were dried over Na₂SO₄ and the solvent was evaporated under reduced pressure. The crude yellow product can be recrystallized from acetonitrile or crystallized by solvent/antisolvent system of water/ethanol or acetone/chloroform to give 3-(2-bromo-3,4-dihydroxyphenyl)-N-(3,4,5-trihydroxy-benzyl)-thioacrylamide in 50-60% yield as yellow crystals. ¹H NMR (300 MHz, CDCl₃): δ 4.77 (d, 2H, J=5.2 Hz, CH₂N), 6.43 (s, 2H, aromatic), 6.86 (d, 1H, J=8.4 Hz, aromatic), 7.01 (d, 1H, J=15.2 Hz, alkene), 7.16 (d, 1H, J=8.4 Hz, aromatic), 8.27 (d, 1H, J=15.2 Hz, alkene), 8.99 (br.s., 1H, NH).

### Example 2: Exposure of the Trans Isomer of 3-(2-bromo-3,4-dihydroxy-phenyl)-N-(3,4,5-trihydroxy-benzyl)-thioacrylamide to UV Light—¹H NMR Analysis

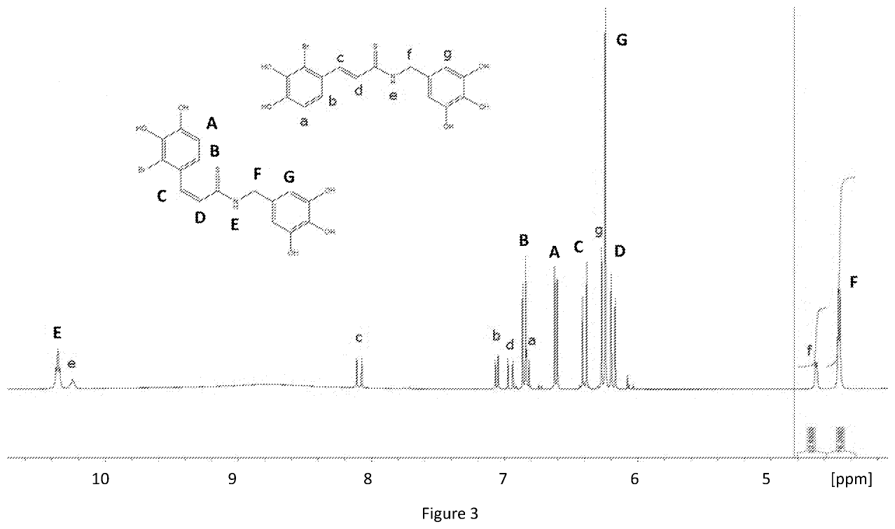

A sample of substantially pure trans isomer of 3-(2-bromo-3,4-dihydroxy-phenyl)-N-(3,4,5-trihydroxy-benzyl)-thioacrylamide in 10% d6-DMSO in a buffer (pH 7.4) was irradiated with an artificial daylight for 5 hr. Thereafter, the solution was characterized by ¹H-NMR analyses. The solution showed a significant conversion of the substantially pure trans isomer to the cis isomer resulting in a mixture of the trans and cis isomers. A representative ¹H-NMR spectrum of the cis-trans mixture resulting from light exposure is shown in FIG. 3.

Table 1 summarizes the ¹H-NMR chemical shifts obtained for the cis and trans isomers of 3-(2-bromo-3,4-dihydroxy-phenyl)-N-(3,4,5-trihydroxy-benzyl)-thioacrylamide. A clear difference between the two isomers, particularly in the double bond coupling (annotations 8 and 9) can be seen. The J_{HH}-coupling of the cis isomer is 12.3 Hz whereas the coupling of the trans isomer is 15.1 Hz. Thus, differentiating between the two isomers can be performed using ¹H-NMR analysis.

TABLE 1
Atom annotation of the cis and trans isomers of 3-(2-bromo-
3,4-dihydroxy-phenyl)-N-(3,4,5-trihydroxy-benzyl)-thioacrylamide

¹H (δ(ppm), integration, split, J_{HH} (Hz))

| Annotation | Cis | Trans |
|---|---|---|
| 1 | — | 8.0 (OH) |
| 2 | — | 8.8 (OH) |
| 3 | 6.27, 2, s | 6.30, 2, s |
| 4 | — | — |
| 5 | 4.51, 2, d, 5.8 | 4.69, 2, d, 5.63 |
| 6 | 10.36, 1, t, 5.6 | 10.25, 1, t, 5.6 |
| 7 | — | — |
| 8 | 6.20, 1, d, 12.3 | 6.98, 1, d, 15.1 |
| 9 | 6.42, 1, d, 12.3 | 8.11, 1, d, 15.1 |
| 10 | — | — |
| 11 | — | — |
| 12 | — | 10.26 (OH) |
| 13 | — | 9.3 (OH) |
| 14 | 6.63, 1, d, 8.4 | 6.85, 1, d, 8.4 |
| 15 | 6.87, 1, d, 8.4 | 7.08, 1, d, 8.5 |

### Example 3: Preparation of Formulations of the Trans Isomer of 3-(2-bromo-3,4-dihydroxy-phenyl)-N-(3,4,5-trihydroxy-benzyl)-thioacrylamide in HPCD

A formulation (4 L, density 1.15 g/mL) of the trans isomer of 3-(2-bromo-3,4-dihydroxy-phenyl)-N-(3,4,5-trihydroxy-benzyl)-thioacrylamide in 2-hydroxypropyl-β-cyclodextrin (HPCD) was prepared in a 10 L glass double jacket reactor, equipped with a Teflon-coated controlled speed agitator. Particularly, 1,680 grams of HPCD were added to Water for Injection (WFI) (2,040 g) with mixing at 50° C./about 250 RPM. The solution was cooled to room temperature after complete dissolution of the HPCD. 3-(2-bromo-3,4-dihydroxy-phenyl)-N-(3,4,5-trihydroxy-benzyl)-thioacrylamide trans isomer (280 g) was added to the solution and mixed at room temperature under light protection conditions until complete dissolution was achieved.

Additional WFI (600 g) were added to the solution with mixing at room temperature/about 250 RPM and under light protection to wash the inner surface of the reactor and to complete the volume to a total of 2,640 g WFI in the formulation. The bulk solution was filtered and sterilized using sterile and depyrogenic 0.22 μm filtration units into a light-protected sterile container. The formulation was stored in Amber vials protected from light at −20° C. until use.

### Example 4: Exposure of the Trans Isomer of 3-(2-bromo-3,4-dihydroxy-phenyl)-N-(3,4,5-trihydroxy-benzyl)-thioacrylamide to Light—UV Analysis

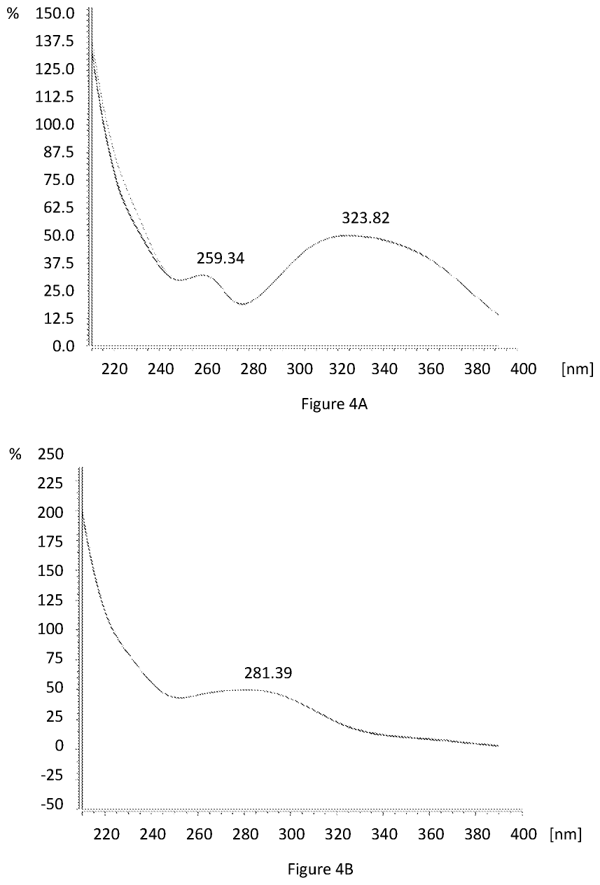

Solutions of 100 mg/mL of the trans isomer of 3-(2-bromo-3,4-dihydroxy-phenyl)-N-(3,4,5-trihydroxy-benzyl)-thioacrylamide dissolved in 600 mg/mL HPCD, were diluted in 0.025% phosphoric acid to a solution of 0.5 mg/mL 3-(2-bromo-3,4-dihydroxy-phenyl)-N-(3,4,5-trihydroxy-benzyl)-thioacrylamide and placed for several days on the bench in clear glass vials exposed to room lighting. The exposure resulted in a conversion of the trans isomer to the cis isomer. The UV spectra of trans and cis isomers are shown in FIGS. 4A and 4B, respectively.

### Example 5: Short Term Stability of Diluted Formulations

The stability of the trans isomer upon exposure to artificial daylight was assessed. The following solutions was prepared:

D5W—(5% w/v dextrose in DDW): 5 g of dextrose were dissolved in 100 ml DDW and filtered using a 0.22 μm filter.
Stock solution X—(1,000 mM solution in DMSO): 4.12 mg of 3-(2-bromo-3,4-dihydroxy-phenyl)-N-(3,4,5-trihydroxy-benzyl)-thioacrylamide were dissolved in 10 μL DMSO. The stock solution was shown to contain 2.3% of the cis isomer and 97.6% of the trans isomer. Stock solution X was then diluted to a concentration of 3.6 mM 3-(2-bromo-3,4-dihydroxy-phenyl)-N-(3,4,5-trihydroxy-benzyl)-thioacrylamide by adding 915 μL D5W and further diluting 300 μL of the latter solution in 600 μL D5W. The diluted solution was divided to two, one kept protected from light at 4° C. (solution X1) and the other was exposed to artificial daylight at room temperature (about 20° C.) for 4 hr (solution X2).
Stock solution Y—(169.9 mM solution in HPCD): 70 mg/mL solution of 3-(2-bromo-3,4-dihydroxy-phenyl)-N-(3,4,5-trihydroxy-benzyl)-thioacrylamide in 420 mg/mL HPCD in water for injection was prepared by dissolving HPCD in the water for injection and adding 3-(2-bromo-3,4-dihydroxy-phenyl)-N-(3,4,5-trihydroxy-benzyl)-thioacrylamide as described in Example 3. The stock solution was shown to contain 0.3% of the cis isomer and 99.1% of the trans isomer. The stock solution was then diluted to a concentration of 3.6 mM 3-(2-bromo-3,4-dihydroxy-phenyl)-N-(3,4,5-trihydroxy-benzyl)-thioacrylamide by adding 42.4 μL of stock solution Y to 1,958 μL D5W. The diluted solution was divided to two, one kept protected from light at 4° C. (solution Y1) and the other was exposed to artificial daylight at room temperature (about 20° C.) for 4 hr and then wrapped in an aluminum foil and further kept at 4° C. (solution Y2).
Stock solution Z—(10 mM solution in DMSO): 4.12 mg of 3-(2-bromo-3,4-dihydroxy-phenyl)-N-(3,4,5-trihydroxy-benzyl)-thioacrylamide were dissolved in 1 mL DMSO. The stock solution was shown to contain 0.4% of the cis isomer and 99.5% of the trans isomer. The stock solution was then diluted to a concentration of 3.6 mM 3-(2-bromo-3,4-dihydroxy-phenyl)-N-(3,4,5-trihydroxy-benzyl)-thioacrylamide by adding 300 μL of stock solution Z to 600 μL D5W. The diluted solution was divided to two, one kept protected from light at 4° C. (solution Z1) and the other was exposed to artificial daylight at room temperature (about 20° C.) for 4 hr, and then wrapped in an aluminum foil and further kept at 4° C. (solution Z2).

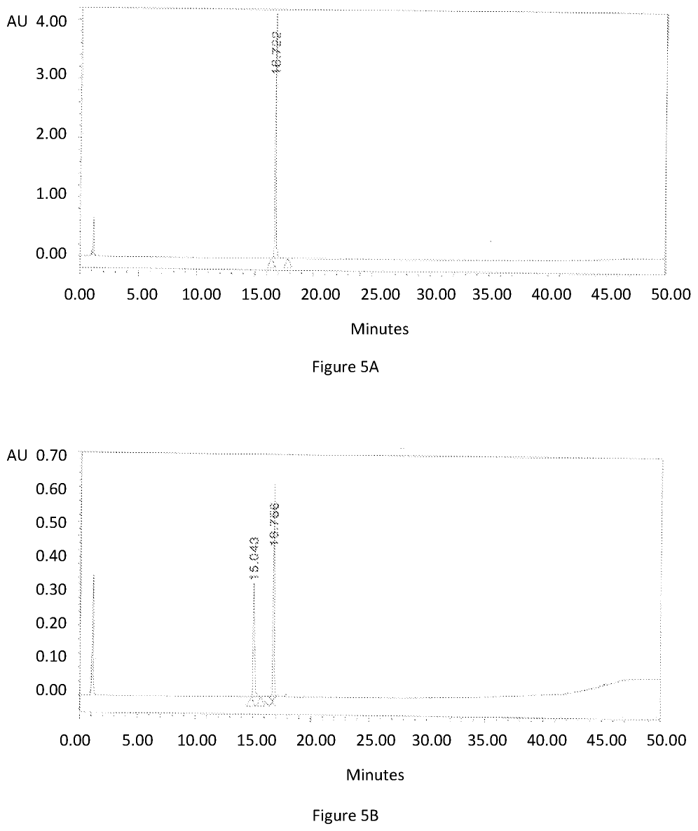

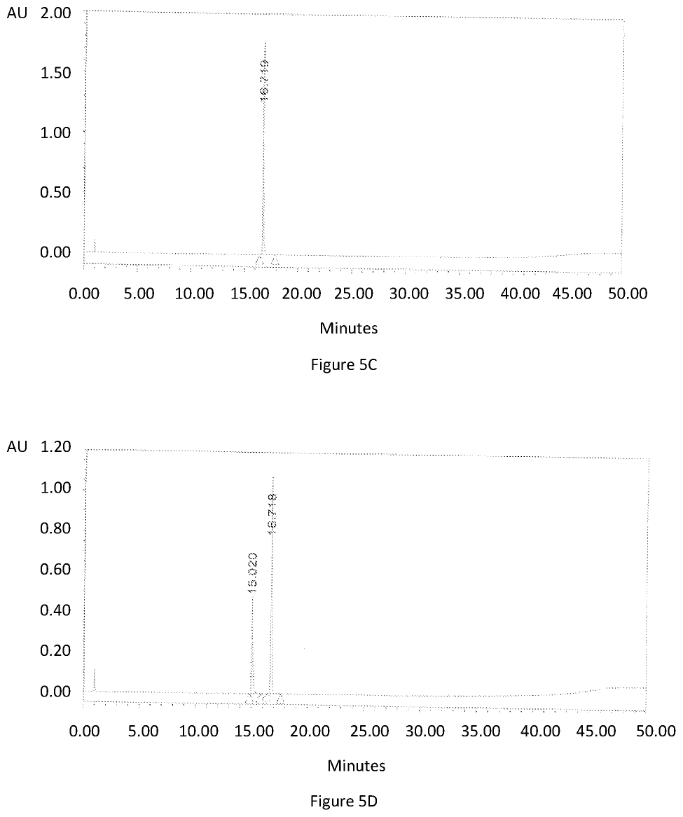

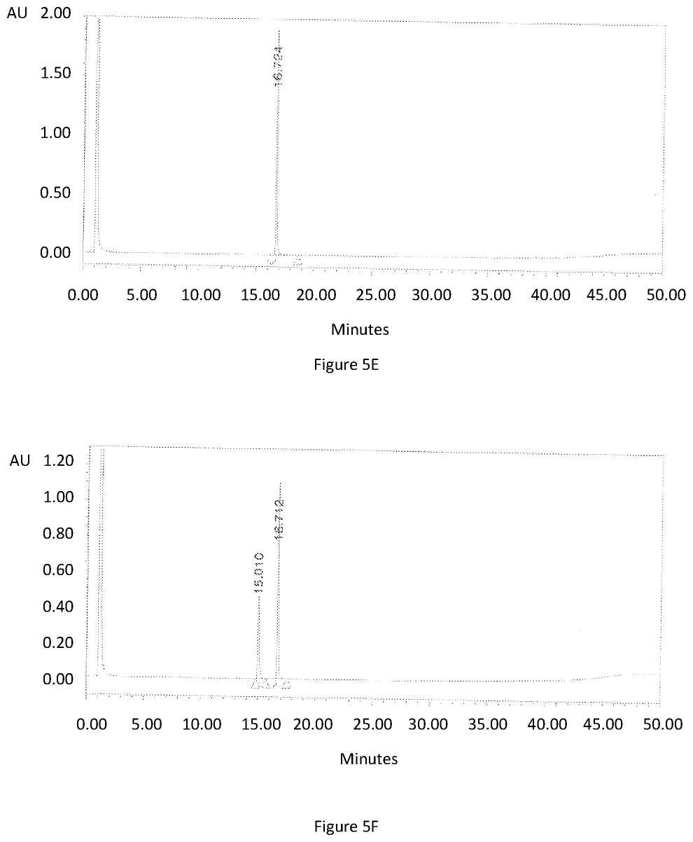

HPLC analysis of the various solutions for the determination of the cis to trans ratios was performed. The results are presented in Table 2 and FIGS. 5A-5F. In particular, FIGS. 5A, 5C, and 5E show the HPLC chromatograms of solutions that were protected from light (solution X1, solution Y1, and solution Z1, respectively) and FIGS. 5B, 5D, and 5F show the HPLC chromatograms of solutions that were exposed to light for 4 hours (solution X2, solution Y2, and solution Z2, respectively).

TABLE 2
HPLC analysis of solutions

Purity by HPLC, % (Area, mln)

| Sample | Cis-isomer RRT0.90 | Trans-isomer RRT1.0 |
|---|---|---|
| Solution X1 | 0.4 | 99.5 |
| Solution X2 | 41.8 | 55.6 |
| Solution Y1 | 0.3 | 99.2 |
| Solution Y2 | 37.1 | 62.0 |
| Solution Z1 | 0.7 | 99.2 |
| Solution Z2 | 35.5 | 64.1 |

The results show that a significant conversion of a substantially pure trans isomer (Rt˜16.7 minutes) to the cis isomer (Rt˜15.0 minutes) is induced by light exposure.

An additional assessment of the stability of the trans isomer upon exposure to artificial daylight was made. Solutions containing 100% of the trans isomer at 1 mM concentration were exposed to 125 lux light (1 lux of light equals 1 lumen per square meter (lm/m²)), and the conversion to the cis isomer was determined by HPLC at different time points. The results are summarized in Table 3.

TABLE 3
Trans to cis conversion upon light exposure

| Time | | |
|---|---|---|
| [hours] | % trans | % cis |
| 0 | 100 | 0 |
| 1 | 50.5 | 49.5 |
| 2 | 36.3 | 63.7 |
| 4 | 12.3 | 87.7 |
| 5 | 8.5 | 91.5 |
| 6 | 7.9 | 92.1 |

Thus, approximately 50% conversion of the trans isomer to the cis isomer was detected following 1 hour exposure to 125 lux light at 4° C. Conversion was further progressed to 64% cis following 2 hours of exposure, and over 90% cis following 5 hours of exposure.

### Example 6: Anti-Proliferative Activity of 3-(2-bromo-3,4-dihydroxy-phenyl-N-3,4,5-trihydroxy-benzyl)-thioacrylamide Isomers

### Test Formulation

A stock solution of 3-(2-bromo-3,4-dihydroxy-phenyl)-N-(3,4,5-trihydroxy-benzyl)-thioacrylamide was prepared by dissolving 1.5 mg of the compound in 1.8 mL ethanol in a light-protected tube to yield a 2 mM solution. 1.48 mL of the solution was then added to 2.52 mL of sterile DDW to provide a concentration of 0.74 mM (0.3 mg/mL) in 37% EtOH in water. The solution was divided into 4 tubes, tube #0 was kept in the dark at 4° C., wrapped in an aluminum foil, while the other tubes were exposed to daylight at R.T. for 1 h, 4 h, and 24 h, labelled as #1, #4, and #24, respectively. The solutions were used for the cell proliferation study and further kept frozen for chemical analysis, wrapped in an aluminum foil. The content of the cis and the trans isomers in each solution was analyzed with extra precaution from light using HPLC.

### Cell Culture

A375 (malignant human melanoma) cells were purchased from the ATCC (American Type Culture Collection). Cells were cultured and grown in RPMI 1640 medium supplemented with 10% fetal bovine serum, 1% glutamine, 1% penicillin, and 1% streptomycin (complete medium), in a light protected, humidified atmosphere of 95% air and 5% CO₂ at 37° C.

### Experimental Design

The design of the study included 5 testing systems of human melanoma A375 cells, treated with solutions at final concentrations of 0, 0.1, 0.3, 1, 3, and 10 μM, in 5-plicates (5 wells per concentration).

Human melanoma A375 cells were seeded in 96 well-plates (1,500 cells/well) in a complete medium (180 μL/well). Prior to treatment of the cells, the solutions (0.74 mM, tubes #0, #1, #4, #24) were diluted with 5% EtOH in sterile water to concentrations of 0, 1, 3, 10, 30 and 100 μM (×10 of the final concentrations in the cell proliferation assays).

A day following seeding (Day 0) the 3-(2-bromo-3,4-dihydroxy-phenyl)-N-(3,4,5-trihydroxy-benzyl)-thioacrylamide solutions were added at the concentrations indicated above (20 μL/well) for 3 days. Following 3 days of treatment, cells were fixed with glutaraldehyde and cell viability was quantified using methylene blue.

An additional plate of A375 cells was fixed on Day 0 with glutaraldehyde, and stained with methylene blue together with all other plates, to quantify cell viability on treatment initiation. Analysis of the results was performed by calculating the % OD of the control (no drug), and IC₅₀ values were calculated using the Prism software.

The statistical significance of the difference between the treatments was evaluated by One-Way ANOVA with a Post Hoc Tukey's HSD Test at the 0.3 μM concentration (around the IC₅₀ values).

### Results

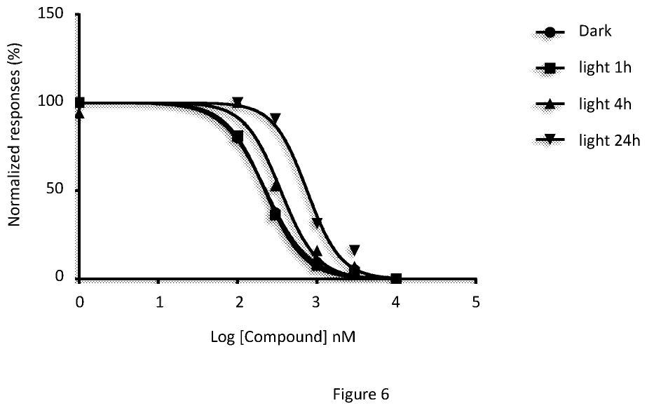

Exposure of 0.3 mg/mL (0.74 mM) of 3-(2-bromo-3,4-dihydroxy-phenyl)-N-(3,4,5-trihydroxy-benzyl)-thioacrylamide solution to daylight for 1, 4, and 24 hours yielded solutions with increased ratios of cis to trans isomers. Significant inhibition of A375 cell proliferation was detected in all ratios of the isomers, demonstrating a dose dependent effect (Table 4 and FIG. 6).

TABLE 4
Percent of cis and trans isomers in solutions vs. IC₅₀ values

| | IC₅₀ | IC₅₀ (nM) | | |
|---|---|---|---|---|
| Preparation | (nM) | Normalized | trans (%) | cis (%) |
| no light (#0) | 226 | 226 | 99 | 1 |
| 1 hr light (#1) | 222 | 220 | 78 | 20 |
| 4 hr light (#4) | 352 | 345 | 53 | 44 |
| 24 hr light (#24) | 753 | 686 | 18 | 73 |

Importantly, while samples containing 80-100% of the trans isomer (no light or 1 hour of light exposure) showed no significant change in IC₅₀ values (226-222 nM), a significant increase in IC₅₀ values at cis to trans ratios of ˜50:50 (#4) was observed (352 nM). The trend of increase in IC₅₀ values was further seen when the cis to trans ratios increased to ˜80:20 after 24 hours of light exposure (753 nM), and even when normalizing the IC₅₀ values based on the overall compound (cis+trans) assay (686 nM). The differences are statistically significant. Accordingly, the results show that the interconversion of the trans isomer to a cis-trans mixture is accompanied by loss of anti-proliferative activity and that a substantially pure trans isomer is a more potent anti-proliferative agent.

The anti-proliferative activity of the substantially pure trans isomer versus a cis-trans mixture is further evaluated using the following cell lines: Lung cancer: NCI-H1975; Head and Neck cancer: SCC-9; Colorectal cancer: HCT116; Sarcoma: SK-ES.1; Hepatocellular cancer: HepG2; Breast cancer: MDA-MB-468 and MDA-MB-231; Multiple Myeloma: MM1S and RPMI-8226; Ovarian cancer: A2780; Gastric cancer: NCI-N87; Epidermoid cancer: A431; Lymphoma: KARPAS; Osteosarcoma: Saos2; Pancreatic cancer: Panc1; Bladder cancer: T24P; Glioblastoma: U138MG; Prostate cancer: DU145; and Leukemia: K562. Cells are exposed to 3-(2-bromo-3,4-dihydroxy-phenyl)-N-(3,4,5-trihydroxy-benzyl)-thioacrylamide solutions at concentrations of 0, 0.1, 0.3, 1, 3, and 10 μM which contain various cis to trans ratios. Cell proliferation and viability are measured by methylene blue or mitochondrial activity assay (e.g. Cell Titer-Glo, WST-1 assays).

### Example 7: Partition Coefficient

The octanol:water partition coefficients of the trans and cis isomers of 3-(2-bromo-3,4-dihydroxy-phenyl)-N-(3,4,5-trihydroxy-benzyl)-thioacrylamide were determined. The partition coefficient is an important physical characteristic of a substance that is known to correlate with its ability to be absorbed by tissues. The partitioning of a pharmaceutical substance provides an assessment of its lipophilicity which is a major factor determining the absorption of the substance, its distribution in the body, penetration across vital membranes and biological barriers, metabolism and excretion (ADME properties). Log P (logarithm of the partition coefficient) is an important factor governing passive membrane partitioning with a direct correlation between Log P and permeability. Furthermore, bioavailability highly depends on the solubility, permeability, and clearance which, in turn, depend on lipophilicity. Log P in the range of zero to three is considered to afford good bioavailability of an active substance.

### Protocol

A solution of 10 mM of the trans isomer in DMSO was prepared and diluted 10-fold in water. A solution of the cis isomer was obtained by exposing the trans isomer solution (5 mL) to 125 lux light at 4° C. for 9 hours resulting in a solution containing more than 90% of the cis isomer. Partitioning in octanol:water was performed as follows: 20 μL of each solution were added to 80 μL of water (saturated by octanol, containing 1 mM of ascorbic acid) and 100 μl of octanol (saturated by water). The obtained solutions were stirred for 1 min and left for incubation at RT for 24 hours. Then, each phase was analyzed by HPLC. The concentration (C) of each isomer was determined using a calibration curve, based on the injection volume of each phase and a response factor of 1 (previously measured).

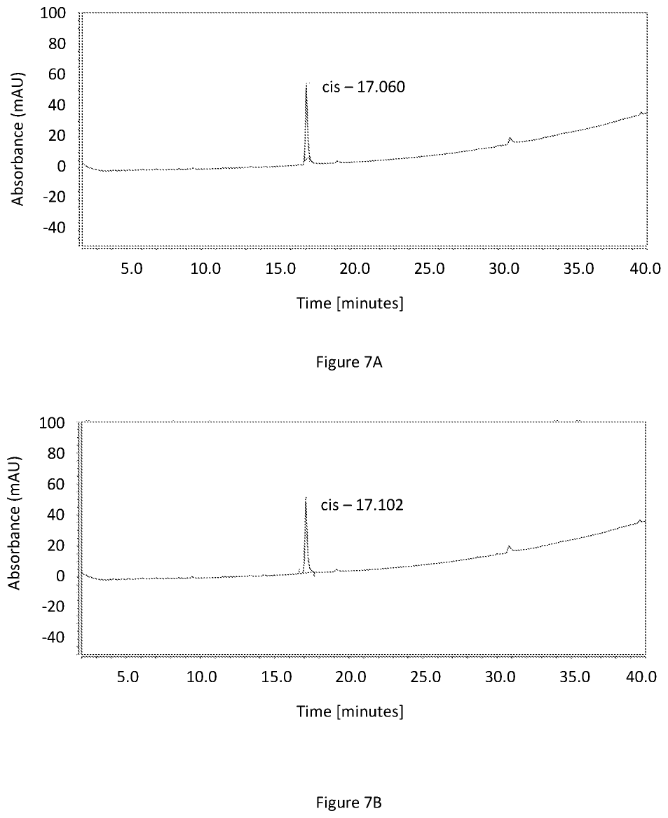

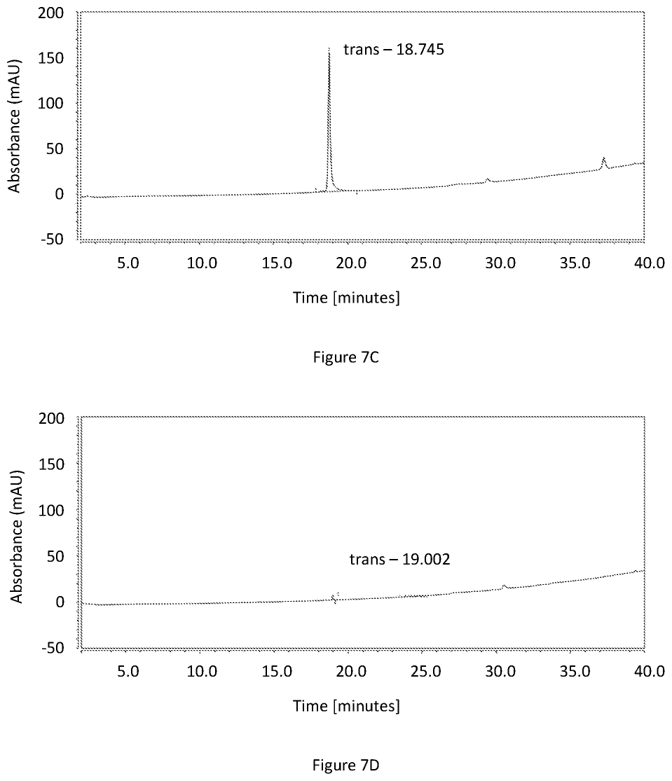

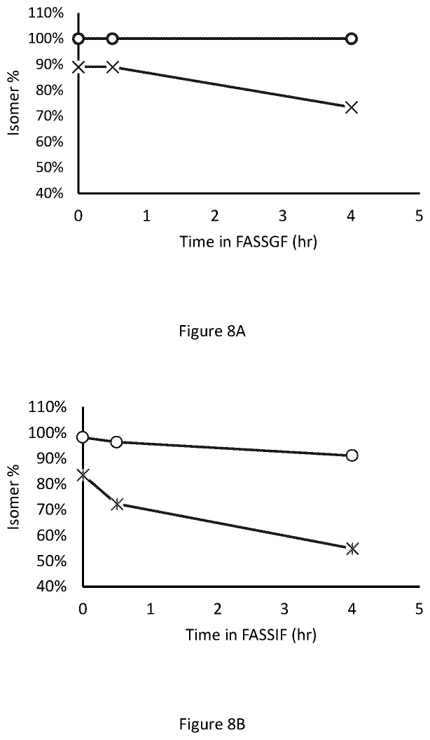

FIGS. 7A and 7B show the HPLC chromatograms of the cis isomer (˜0.1 mM, 1% DMSO) in an octanol phase and a water phase, respectively. The calculated log P (log(C_{octanol}/C_{water})) was −0.18. FIGS. 7C and 7D show the HPLC chromatograms of the trans isomer (0.1 mM, 1% DMSO) in an octanol phase and a water phase, respectively. For all said analyses a volume of 13 μL was injected to the HPLC. Since the level of the trans isomer in the water phase was below the limit of quantification (BLQ), a higher volume (35 μL) of the water phase was analyzed in a repeated experiment and used for the calculation of the Log P value using a calibration curve while taking into account the analyzed volume. The calculated log P (log(C_{octanol}/C_{water})) for the trans isomer was 0.86.

Thus, the log P value of the trans isomer of 3-(2-bromo-3,4-dihydroxy-phenyl)-N-(3,4,5-trihydroxy-benzyl)-thioacrylamide is indicative of its improved bioavailability as compared to the cis isomer.

### Example 8: Stability in Biorelevant Fluids

The stability of the trans and cis isomers of 3-(2-bromo-3,4-dihydroxy-phenyl)-N-(3,4,5-trihydroxy-benzyl)-thioacrylamide in biorelevant fluids was assessed, including Fasted State Simulated Intestinal Fluid (FaSSIF) and Fasted State Simulated Gastric Fluid (FaSSGF).

### Reagents

FaSSIF/FeSSIF/FaSSGF Powder (Biorelevant cat #FFF01)
FaSSIF Buffer Concentrate (Biorelevant cat #FASBUF)
FaSSGF Buffer Concentrate (Biorelevant cat #FASGBUF)

### Protocol

A solution containing the cis isomer of 3-(2-bromo-3,4-dihydroxy-phenyl)-N-(3,4,5-trihydroxy-benzyl)-thioacrylamide was prepared by exposing a 5 mL solution of the 100% trans isomer at a concentration of 1 mM to 125 lux light for 9 hours at 4° C.

FaSSIF and FaSSGF solutions were prepared according to the manufacturer's instructions.

The cis and trans isomers of 3-(2-bromo-3,4-dihydroxy-phenyl)-N-(3,4,5-trihydroxy-benzyl)-thioacrylamide were diluted in either FaSSIF or FaSSGF solutions to a final concentration of 250 μM. Samples were taken at time 0 and after 4 hours. Samples were frozen in liquid nitrogen, and stored frozen until analysis.

The concentrations of the cis and trans isomers at each time point were analyzed by HPLC The results are outlined in Table 5 and are shown in FIGS. 8A (cis x, and trans ∘) and 8B (cis *, and trans ∘).

TABLE 5
Percent of cis and trans isomers in solutions that simulate
gastric fluid (FaSSGF) (A) and intestinal fluids (FaSSIF) (B)

| | 0 h | 0.5 h | 4 h |
|---|---|---|---|
| A | | | |
| Trans in FaSSGF | 100% | 100% | 100% |
| Cis in FaSSGF | 89.05% | 89.07% | 73.47% |
| B | | | |
| Trans in FaSSIF | 98.15% | 96.25% | 90.98% |
| Cis in FaSSIF | 83.49% | 72.21% | 54.87% |

Thus, while the trans isomer remained completely stable in FaSSGF, the concentration of the cis isomer was reduced in ˜16% (FIG. 8A). The same trend was observed in a solution simulating intestinal fluids, while the trans isomer reduced in only ˜7%, the cis significantly decreased to ˜55% after 4 hours (FIG. 8B).

### Example 9: Interactions with Water Molecules

The interactions of the trans and cis isomers of 3-(2-bromo-3,4-dihydroxy-phenyl)-N-(3,4,5-trihydroxy-benzyl)-thioacrylamide with water molecules were assessed using Thermogravimetric Analysis (TGA).

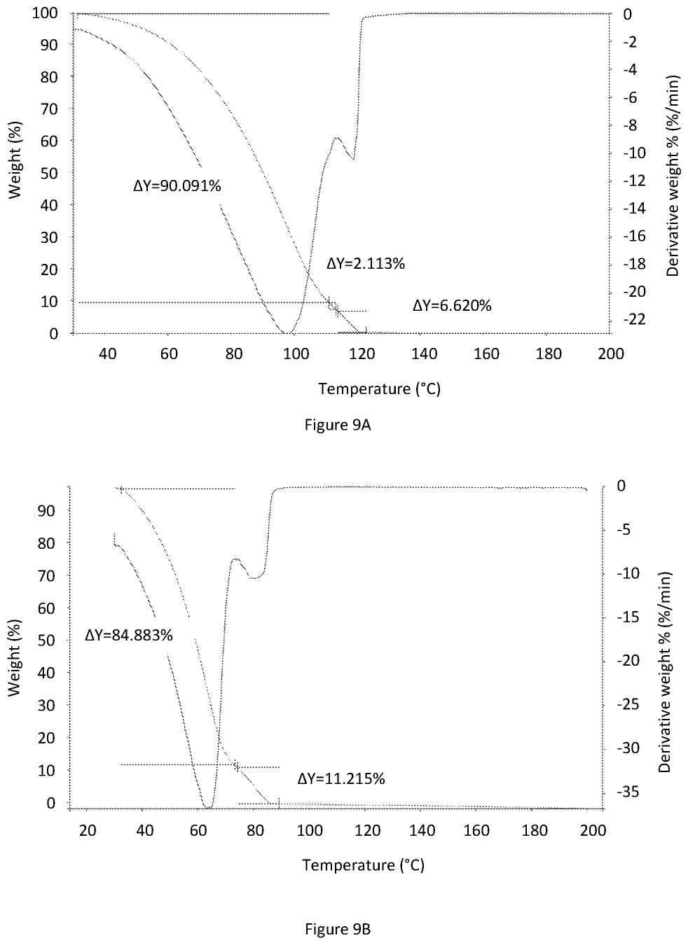

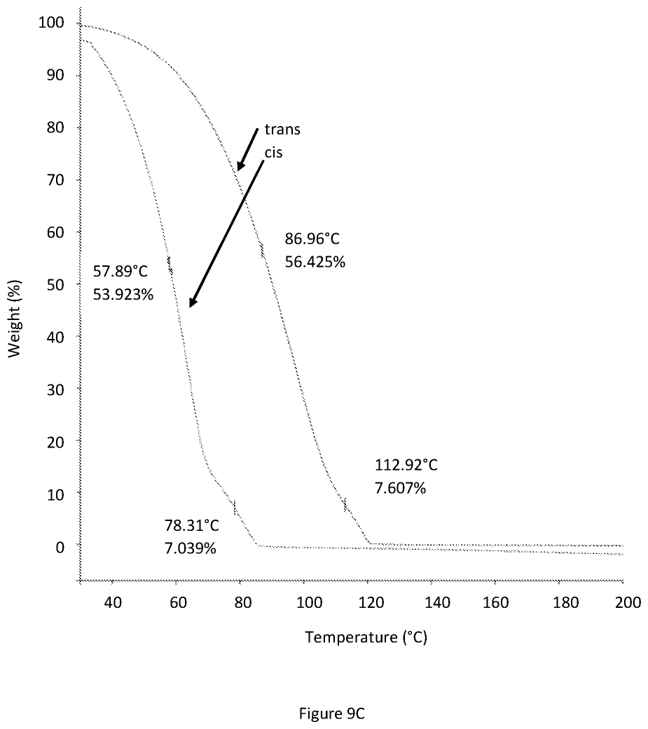

TGA was performed on a Perkin Elmer TGA 800 using Pyris software. A temperature scan from 30° C. to 200° C. at a rate of 10° C./min was performed. The isomers were analyzed at a concentration of 1 mM in water (containing 1% DMSO). The results are outlined in Table 6 and are shown in FIGS. 9A-9C.

TABLE 6
Water evaporation from the cis and trans isomers

| | Trans isomer | Cis isomer |
|---|---|---|
| Temperature of mid evaporation (free water) | 87° C. | 58° C. |
| Maximum evaporation rate (free water) | 23%/min | 37%/min |
| Maximum evaporation rate (bound water) | 12%/min | 12%/min |

Whereas the free water in the cis isomer evaporated at a relatively low temperature, free water evaporation in the trans isomer occurred at a significantly higher temperature including the onset temperature, mid temperature and end temperature. The evaporation rate of the free water also showed significant differences between the cis and trans isomers whereby the free water in the cis isomer evaporated at a much faster rate (37%/min) than the free water in the trans isomer (23%/min). Without being bound by any theory or mechanism of action, the trans isomer seem to have no significant effect on water organization thereby allowing free adsorbed water to behave in a typical solution-like manner. Since the interaction of a molecule with water can correlate with several biological/medicinal parameters such as bioavailability, excretion time and mechanism of dispersal, it is contemplated that the trans isomer would therefore exert an improved stability in an aqueous environment as compared to the cis isomer. Thus, the trans isomer is believed to be more bioavailable than the cis isomer.

While certain embodiments of the invention have been illustrated and described, it is to be clear that the invention is not limited to the embodiments described herein. Numerous modifications, changes, variations, substitutions and equivalents will be apparent to those skilled in the art without departing from the spirit and scope of the present invention as described by the claims, which follow.

1. A substantially pure trans isomer of 3-(2-bromo-3,4-dihydroxy-phenyl)-N-(3,4,5-trihydroxy-benzyl)-thioacrylamide having the following structural formula: 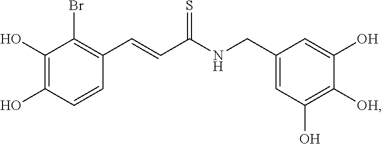 [SMILES: Oc1cc(CNC(=S)/C=C/c2ccc(O)c(O)c2Br)cc(O)c1O]
or a pharmaceutically acceptable salt thereof.

2. The substantially pure trans isomer according to claim 1, which comprises at least 80% by weight of the trans isomer of 3-(2-bromo-3,4-dihydroxy-phenyl)-N-(3,4,5-trihydroxy-benzyl)-thioacrylamide and less than 20% by weight of the cis isomer of 3-(2-bromo-3,4-dihydroxy-phenyl)-N-(3,4,5-trihydroxy-benzyl)-thioacrylamide.

3. The substantially pure trans isomer according to claim 1, which comprises at least 85% by weight of the trans isomer of 3-(2-bromo-3,4-dihydroxy-phenyl)-N-(3,4,5-trihydroxy-benzyl)-thioacrylamide and less than 15% by weight of the cis isomer of 3-(2-bromo-3,4-dihydroxy-phenyl)-N-(3,4,5-trihydroxy-benzyl)-thioacrylamide.

4. The substantially pure trans isomer according to claim 1, which comprises at least 90% by weight of the trans isomer of 3-(2-bromo-3,4-dihydroxy-phenyl)-N-(3,4,5-trihydroxy-benzyl)-thioacrylamide and less than 10% by weight of the cis isomer of 3-(2-bromo-3,4-dihydroxy-phenyl)-N-(3,4,5-trihydroxy-benzyl)-thioacrylamide.

5. The substantially pure trans isomer according to claim 1, which comprises at least 95% by weight of the trans isomer of 3-(2-bromo-3,4-dihydroxy-phenyl)-N-(3,4,5-trihydroxy-benzyl)-thioacrylamide and less than 5% by weight of the cis isomer of 3-(2-bromo-3,4-dihydroxy-phenyl)-N-(3,4,5-trihydroxy-benzyl)-thioacrylamide.

6. The substantially pure trans isomer according to claim 1, which comprises at least 97% by weight of the trans isomer of 3-(2-bromo-3,4-dihydroxy-phenyl)-N-(3,4,5-trihydroxy-benzyl)-thioacrylamide and less than 3% by weight of the cis isomer of 3-(2-bromo-3,4-dihydroxy-phenyl)-N-(3,4,5-trihydroxy-benzyl)-thioacrylamide.

7. A pharmaceutical composition comprising, as an active ingredient, the substantially pure trans isomer according to claim 1 and a pharmaceutically acceptable carrier or excipient.

8. The pharmaceutical composition according to claim 7 in the form of a solution, suspension or emulsion.

9. The pharmaceutical composition according to claim 7, wherein the pharmaceutically acceptable carrier or excipient is hydroxypropyl-β-cyclodextrin (HPCD).

10. The pharmaceutical composition according to claim 9, wherein the weight ratio between the substantially pure trans isomer of 3-(2-bromo-3,4-dihydroxy-phenyl)-N-(3,4,5-trihydroxy-benzyl)-thioacrylamide or a pharmaceutically acceptable salt thereof and the HPCD is from about 1:1 to about 1:12.

11. The pharmaceutical composition according to claim 7 which is light-protected.

12. The pharmaceutical composition according to claim 11, which is held in an apparatus which is impermeable to visible light, wherein the apparatus has light transmission in the visible wavelength range of less than about 20; or which is stored in a storage container which is impermeable to visible light, wherein the storage container has light transmission in the visible wavelength range of less than about 20%; or which is provided in a kit suitable for intravenous administration which is impermeable to visible light, wherein the kit has light transmission in the visible wavelength range of less than about 20%.

13. (canceled)

14. (canceled)

15. The pharmaceutical composition according to claim 11, which is held in an apparatus which is impermeable to UV light, wherein the apparatus has light transmission in the UV wavelength range of less than about 20%; or which is stored in a storage container which is impermeable to UV light, wherein the storage container has light transmission in the UV wavelength range of less than about 20%; or which is provided in a kit suitable for intravenous administration which is impermeable to UV light, wherein the kit has light transmission in the UV wavelength range of less than about 20%.

16. (canceled)

17. (canceled)

18. (canceled)

19. (canceled)

20. (canceled)

21. (canceled)

22. A method of treating cancer, the method comprising administering to a subject in need thereof the substantially pure trans isomer according to claim 1 or a pharmaceutical composition comprising said isomer and a pharmaceutically acceptable carrier or excipient.

23. The method according to claim 22, wherein the cancer is selected from the group consisting of head and neck cancer, sarcoma, multiple myeloma, ovarian cancer, breast cancer, kidney cancer, stomach cancer, hematopoietic cancers, lymphoma, leukemia, lung carcinoma, melanoma, glioblastoma, hepatocarcinoma, esophageal cancer, gastroesophageal junction cancer, prostate cancer, pancreatic cancer, and colon cancer.

24. The method according to claim 22 comprising co-administering the substantially pure trans isomer or the pharmaceutical composition in combination with an anti-cancer agent.

25. The method according to claim 24, wherein the anti-cancer agent comprises at least one of (i) a modulator of a protein kinase (PK) selected from an Epidermal Growth Factor Receptor inhibitor (EGFR inhibitor) and EGFR antibody; (ii) an inhibitor of mammalian target of rapamycin (mTOR); (iii) a mitogen-activated protein kinase (MEK) inhibitor; (iv) a mutated B-Raf inhibitor; (v) a chemotherapeutic agent; and (vi) an immunotherapy agent comprising an antibody against programmed cell death 1 (PD-1) protein, programmed cell death protein 1 ligand (PD-L1), cytotoxic T-lymphocyte-associated protein 4 (CTLA4), or a combination thereof.

26. (canceled)

27. (canceled)

28. (canceled)

29. (canceled)

30. A method of preventing the conversion of the substantially pure trans isomer according to claim 1 to the cis isomer, the method comprising maintaining the substantially pure trans isomer in a light-protected apparatus or container.

31. The method according to claim 30, wherein the light-protected apparatus or container is substantially impermeable to light in the UV-VIS wavelength range.
